(19) United States
(12) Patent Application Publication
(10) Pub. No.: US 2012/0283787 A1
(43) Pub. Date: Nov. 8, 2012

(54) PEDICLE SCREWS AND METHODS OF USE

(75) Inventors: Hansen A. Yuan, Naples, FL (US); Peter J. Slate, Paradise Valley, AZ (US)

(73) Assignee: Bonovo Orthopedics, Inc., Scottsdale, AZ (US)

(21) Appl. No.: 13/504,743

(22) PCT Filed: Oct. 28, 2010
(86) PCT No.: PCT/US10/54449

§ 371 Date: Jul. 19, 2012

Related U.S. Application Data
(60) Provisional application No. 61/255,792, filed on Oct. 28, 2009.

(51) Int. Cl. A61B 17/86; A61B 17/88

(52) U.S. Cl. 606/305

(57) ABSTRACT

A pedicle screw configured to secure a rod is disclosed. In some embodiments, the pedicle screw is provided with a shaft, a yoke, a cap, a saddle and at least one separating device. The shaft has a forward end, a rear end and a longitudinal axis, the forward end being configured for insertion into a bone. The yoke is attached to the rear end of the shaft and has an interior wall nearest the shaft configured to bear against a rod inserted into the yoke. The cap is configured to engage an end of the yoke opposite the shaft. The saddle has a first side configured to mate with the cap within the yoke, and has an opposite second side configured to bear against the rod. The at least one separating device is coupled between the cap and the saddle, and is configured to urge the cap and the saddle apart from one another. With this arrangement, the yoke, the cap, the saddle and the at least one separating device cooperate to urge the rod against the interior wall of the yoke when the at least one separating device urges the cap and the saddle apart. Methods of securing a rod to a pedicle screw are also disclosed.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The present invention relates to implants, systems and methods for treating various types of orthopedic pathologies, and in particular relates to attachment of spinal implants to vertebral bodies.

### SUMMARY OF THE DISCLOSURE

Aspects of this invention relate to prostheses for use in fusion, stabilization and/or motion preservation of the spine, and in particular to pedicle screws which are capable of being fixed in a pedicle of a vertebral body.

Internal spinal fixation systems are used in the treatment of spinal fractures and to correct spinal deformities. In general such systems involve screws having long shafts, which are inserted into the pedicles and which are then clamped to rods or plates which often run roughly parallel to the spine. The screws serve as anchors on the spine and by adjusting the position of the shafts of the screw relative to the rods or plates, the spine, or a section thereof; may be immobilized in the desired configuration. Various devices are known for fixing the spine and/or sacral bone adjacent the vertebra, as well as attaching devices used for fixation, including: U.S. Pat. Nos. 4,887,596, 6,811,567, 6,619,091, 6,290,703, 5,782,833, 5,738,585, 6,547,790, 6,638,321, 6,520,963, 6,074,391, 5,569,247, 5,891,145, 6,090,111, 6,451,021, 5,683,392, 5,863,293, 5,964,760, 6,010,503, 6,019,759, 6,540,749, 6,077,262, 6,248,105, 6,524,315, 5,797,911, 5,879,350, 5,885,285, 5,643,263, 6,565,565, 5,725,527, 6,471,705, 6,554,843, 5,575,792, 5,688,274, 5,690,630, 6,022,350, 4,805,602, 5,474,555, 4,611,581, 5,129,900, 5,741,255, 6,132,430; and U.S. Patent Publication No. 2002/0120272.

The manner in which pedicle screws are attached to rods has been the subject of much thought and there are a wide variety of clamps and brackets for achieving this end. However, prior proposals have involved devices which are complicated in construction and excessively bulky; so much so that it has been difficult if not impossible to attach the rod/screw assembly to adjacent vertebrae. Moreover, with many devices the rod must be secured to one or more screws before all the screws are inserted. In some devices the rod is threaded making a change of relative position of the rod and the screws awkward.

Pedicle screws are disclosed herein which are simple, compact enough to be used on adjacent vertebrae and which provide greater contact area with the rod compared to prior art devices.

In accordance with the disclosure, a pedicle screw comprises a shaft, threaded at one end for insertion into bone and clamping means comprising a yoke or trough adapted to receive a rod at the other end. The yoke is configured to receive a block or panel for clamping an inserted rod against the bottom of the yoke.

In some embodiments, the block is slidingly mounted in the yoke and has at least one screw mechanism for pressing the rod against the bottom of the yoke. The block may comprise two parts that can be urged apart by the screw mechanism when clamping the rod against the yoke.

In some embodiments, the yoke is provided with grooves and the block has mating grooves which dovetail with the grooves in the yoke allowing the block to slide into the yoke from the side. A socket and a matching projection may be provided in the yoke and block so that when the block is inserted in the yoke the block is prevented from sliding in the yoke.

A spinal prosthesis assembly comprising a pedicle screw as described, in combination with a rod for insertion in the yoke of the screw is also disclosed.

### BRIEF DESCRIPTION OF THE DRAWINGS

Aspects of the invention will be described more fully in connection with the accompanying drawings in which:

FIG. 1 is an exploded perspective view of a spinal prosthesis assembly using a pedicle screw according to aspects of the invention.

FIG. 2 is a side view of a pedicle screw according to aspects of the invention.

FIG. 3 is an end view of the screw of FIG. 2.

FIG. 4 is a view in side elevation showing details of the block shown in FIGS. 1, 2 and 3.

FIG. 5 is an end view of the block of FIG. 4 showing the dovetail slot for engagement with the yoke.

FIG. 6 is a schematic posterior view of a portion of a vertebral column showing the use of pedicle screws in spinal fixation assemblies according to aspects of the invention.

FIG. 7 is a perspective view showing the proximal end of another exemplary embodiment of a pedicle screw.

FIG. 8 is an enlarged cross-sectional view showing a portion of block 46 of FIG. 7.

FIG. 9 is a perspective view showing the proximal end of another exemplary embodiment of a pedicle screw.

FIG. 10 is an enlarged cross-sectional view showing a portion of block 46′ of FIG. 9.

FIG. 11A is a top plan view showing a “tulip” portion of another exemplary embodiment of a pedicle screw.

FIG. 11B is a side cross-sectional view showing the tulip of FIG. 11A.

FIG. 11C is a side view showing a set screw configured to mate with the tulip of FIGS. 11A and 11B.

FIG. 11D is a top plan view showing the set screw of FIG. 11C.

FIG. 11E is a side view showing a polyaxial screw configured for use with the tulip of FIGS. 11A and 11B.

FIG. 11F is a side view showing a sagital screw configured for use with the tulip of FIGS. 11A and 11B.

### DETAILED DESCRIPTION

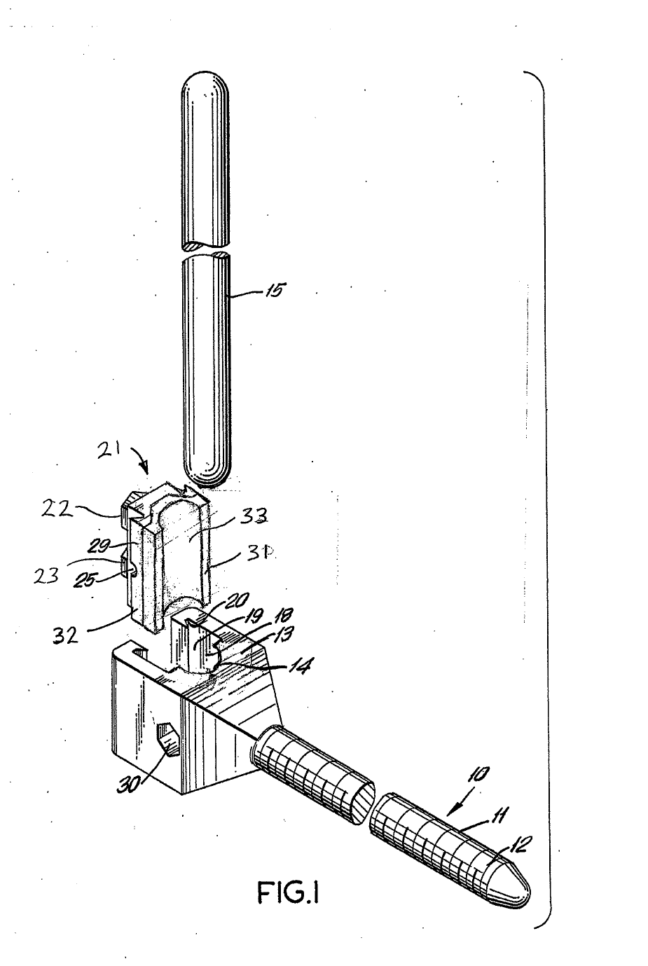

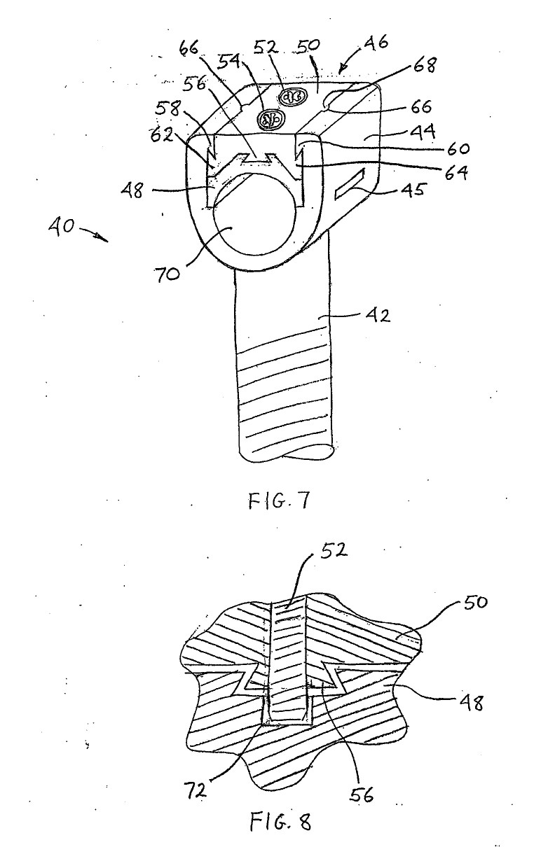

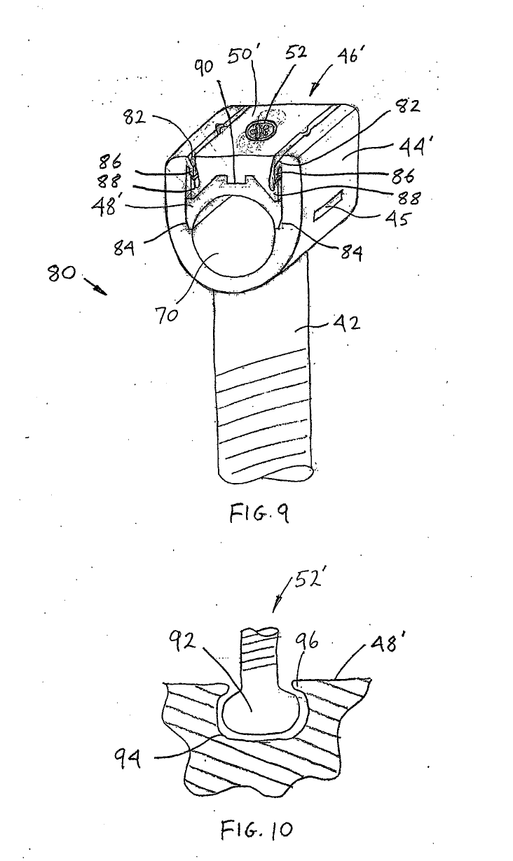

Referring to the drawings, FIG. 1 shows a screw according to aspects of the invention. The screw, indicated generally as 10, comprises a shaft 11 which is threaded at one end as at 12 and preferably somewhat pointed for introduction into the bone. At the opposite end is a yoke or trough 13. The forward end of the yoke is curved as at 14 to receive a rod 15. The rod 15 may be smooth, i.e., unthreaded. The side walls 18 of the yoke are provided with grooves or slots 19 having rear undercut portions 20, for receiving a panel or block 21. As shown in FIGS. 4 and 5, the block 21 has lands 29 with matching undercut portions 26 which form a dovetail joint with the undercut portions of the yoke.

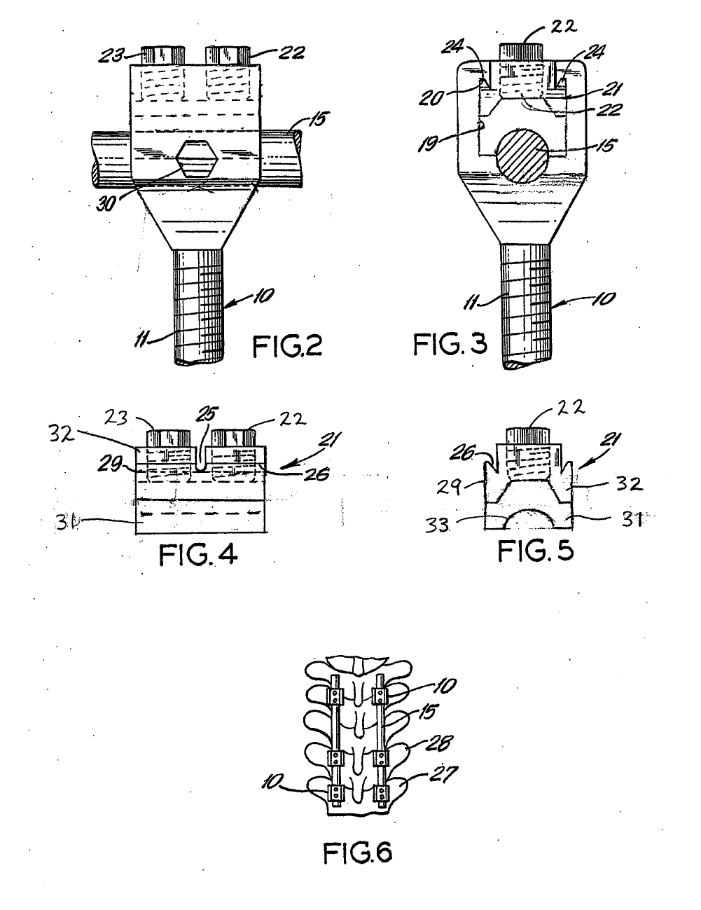

In some embodiments, block 21 comprises two or more pieces that may be interlocked to form one assembly. In the embodiment shown in FIGS. 1-6, block 21 includes saddle 31 and cap 32. Saddle 31 may include a half-bore 33 as best seen in FIGS. 1 and 5 for increased contact with rod 15. In this embodiment, cap 32 includes the previously described undercut portions 26 for mating with yoke 13.

Cap 32 is provided with two set screws 22 and 23 which, as shown in FIG. 3, can be advanced through cap 32 to contact saddle 31. The outer ends of the set screws 22, 23 may be given external hexagonal heads, internal hexagonal sockets, or other keyed features so that they may be operated by a suitable wrench. The thickness of the block 21 may be considerably less than the depth of the yoke 19, so that it will fit loosely in the yoke. When screws 22 and 23 are tightened, saddle 31 and cap 32 are pushed apart. This causes undercut portions 26 on cap 32 to bear against undercut portions 20 on yoke 13, and urges the half-bore 33 of saddle 31 against rod 15. This in turn pushes rod 15 against curved trough 14 in the bottom of yoke 13, thereby locking rod 15 to yoke 13 and preventing relative movement. Because saddle 31 bears against rod 15 instead of screws 22 and 23 bearing against rod 15 directly, rod 15 is engaged with significantly more surface area.

The interior of the undercut portions 20 of the slots 19 may be provided with a small projection 24 (FIG. 3) and a matching socket 25 (FIG. 4) is provided in the block 21 so that when the screws 22, 23 are advanced and press saddle 31 against a rod, forcing the block back against the rear part of the slots 19, the projections 24 will seat in sockets 25 preventing sliding of the block in the socket.

In use the screw may be inserted into the pedicle using Kirschner wires to provide guide holes according to conventional teaching. Sockets 30 as shown, or similar features, may be provided in the side walls of the screw yoke for aid in manipulating the screw. After two or more of the screws have been positioned at the points desired, a rod may be inserted through the open rear ends of the yokes of the screws. The blocks 21 are then dropped into the slots 19 and the set screws 22, 23 tightened. Saddles 31 and caps 32 may be slid into slots 19 together in mating pairs or may be slid into place individually.

FIG. 6 shows a sequence of thoracic vertebrae to which two fixation rods using pedicle screws according to aspects of the invention have been applied. The lateral dimensions of the yoke end of the screw may be quite small; on the order of 12 mm wide, 10 mm deep and 12 mm tall, for example. It is therefore possible, as shown in FIG. 6, to insert screws according to the invention in adjacent vertebrae 27, 28 without interference.

FIG. 7 shows another exemplary embodiment of a pedicle screw 40 constructed according to aspects of the present invention. Screw 40 includes a threaded shank 42 with a generally U-shaped yoke 44 formed at its proximal end. Slotted recesses 45 may be provided in opposite sides of yoke 44 for receiving a tool to assist in installing screw 40 into a bone. A two-piece block 46 is slibably received in yoke 44. Block 46 comprises saddle 48, cap 50, and two set screws 52 and 54. Set screws 52 and 54 are threadably engaged through cap 50 and abut against the top of saddle 48. In some embodiments, set screws 52 and 54 may be generally flush with the top surface of cap 50 as shown, or recessed therein. In other embodiments, a cam device or devices, such as a quarter-turn or half-turn rotating member, may be used as a separating device instead of the set screws to urge saddle 48 and cap 50 apart. A sliding dovetail joint 56 may be formed between saddle 48 and cap 50 as shown to keep these two parts loosely coupled together so that they may be slid into yoke 44 as a single unit. Undercut portions 58 and 60 may be provided on opposite sides of yoke 44 for receiving mating undercut portions 62 and 64 on cap 50. These undercut portions retain block 46 in yoke 44 in a direction along the longitudinal axis of screw 40, while detents 66 in yoke 44 receive protrusions 68 in cap 50 to retain block 46 in a lateral direction. As with screw 10 shown in FIGS. 1-6, block 46 and yoke 44 of screw 40 cooperate to form a bore 70 for receiving and clamping a crossbar (not shown).

FIG. 8 shows a cross section of a portion of saddle 48 and cap 50 through screw 52 and dovetail joint 56. As shown, a counterbore 72 may be provided in saddle 48 under each screw 52 and 54. Block 46 may be assembled by sliding cap 50 into the dovetail slot of saddle 48 before screws 52 and 54 are advanced into cap 50. Screws 52 and 54 may then be threaded into cap 50 and advanced through the dovetail joint 56 until the tips of screws 52 and 54 enter into counterbores 72 in saddle 48. This arrangement keeps saddle 48 and cap 50 from sliding apart once assembled.

In use screw 40 is inserted into bone, such as the pedicle of a vertebral body as previously described. A rod 15 or other prosthesis crossbar may then be placed in yoke 44. Block 46 (comprising saddle 48, cap 50, and screws 52 and 54) may then be slid into yoke 44 over rod 15 until protrusions 68 snap into detents 66. Screws 52 and 54 may then be tightened, urging cap 50 upward against undercuts 58 and 60 of yoke 44 and urging saddle 48 downward against rod 15. Dovetail joint 56 between cap 50 and saddle 48 is loosely fitting to allow saddle 48 to separate sufficiently from cap 50 to clamp against rod 15.

FIG. 9 shows another exemplary embodiment of a pedicle screw 80 constructed according to aspects of the present invention. Screw 80 is constructed and operates in a similar manner to that of screw 40, however in this embodiment block 46′ is loaded into the top of yoke 44′ instead of into the side of the yoke.

To facilitate the top-loading aspect of screw 80, the upper lead-in edges of yoke 44′ may be provided with beveled surfaces 82 as shown. Similarly, the lower edges of saddle 48′ may also be provided with beveled surfaces 84. The close fitting undercut portions 58, 60, 62 and 64 shown in FIG. 7 for device 40 may be replaced with barbed fingers 86 on yoke 44′ and/or barbed fingers 88 on cap 50′ as shown in FIG. 9 for device 80. This arrangement allows block 46′ to be inserted from the top of yoke 44′. As block 46′ is inserted, features on yoke 44′ flex outwardly and/or features on block 46′ flex inwardly. Once block 46′ is seated within yoke 44′, these features snap back into place and retain the block in the yoke.

In this embodiment, a straight joint 90 may be used to keep saddle 48′ from rotating relative to cap 50′ before block 46′ is inserted into yoke 44′. A single screw 52 as shown may be used in this embodiment or in previous embodiments to press the saddle against the rod.

FIG. 10 shows another example of how the saddle and the cap of any of the above embodiments may be coupled together. The distal end of screw 52′ may be provided with an enlarged ball 92 or similar feature. A mating recess 94 may be provided in saddle 48′ for receiving ball 92. Undercut 96 formed around the opening of recess 94 allows ball 92 to be snapped into place and retained in recess 94. Since the opposite end of screw 52′ is threadably engaged with the cap, the saddle and cap are held together by screw 52′ in this arrangement.

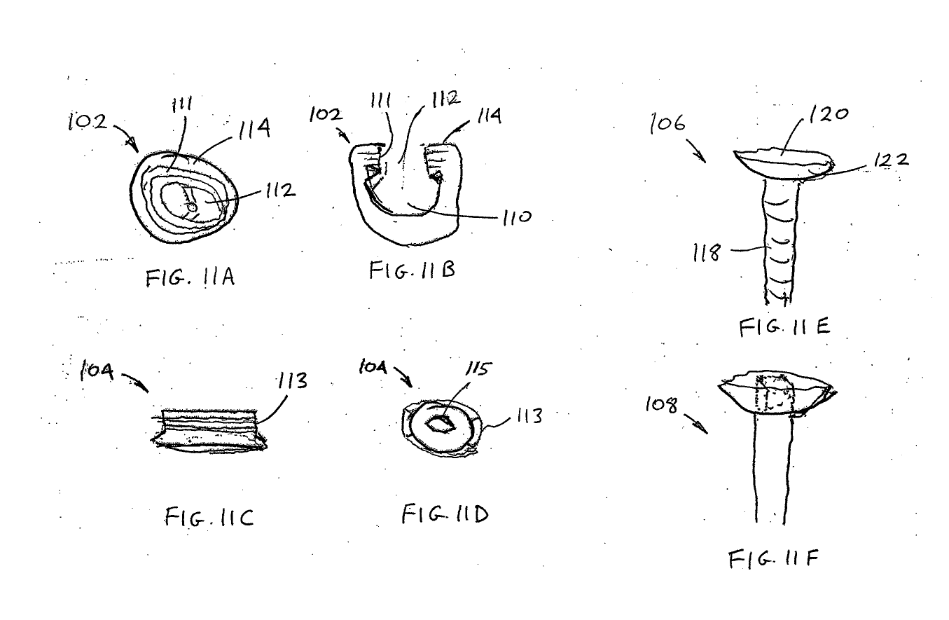

FIGS. 11A-11F shows another exemplary embodiment of a top-loading pedicle screw. FIG. 11A and 11B show a top plan view and a side cross-sectional view, respectively, of a “tulip” portion 102 of the screw. FIGS. 11C and 11D show a side view and a top plan view, respectively, of a set screw 104 configured to engage with tulip 102. FIGS. 11E and 11F show side views of a polyaxial screw 106 and a sagital screw 108, respectively.

Tulip 102 is similar in construction and operation to the yokes of the previously described pedicle screws. Tulip 102 includes a transverse bore 110 therethrough, as shown in FIGS. 11B, for receiving a rod of a spinal prosthesis. Tulip 102 also includes an axial bore 112 through at least the top of tulip 102, as shown in FIGS. 11A and 11B, for receiving set screw 104. The internal surface 111 of tulip 102 formed by axial bore 112 may be threaded, such as with buttress or helical threads. The outer circumference of set screw 104 may be provided with mating threads 113, and the top surface may be provided with a socket 115 for mating with a screw driver tip. In some embodiments, set screw 104 is screwed into tulip 102 to secure a rod passing through transverse bore 110 to tulip 102. In some embodiments, the upper rim 114 of tulip 102 may be segmented or castellated. This arrangement allows portions of the upper rim 114 to flex outwardly as set screw 104 is inserted into axial bore 112. Once set screw 104 is fully seated in tulip 102, these portions of the upper rim 114 snap back into place to lock set screw 104 in place. In some embodiments, portions of the circumference of set screw 104 are removed to match the castellated portions of the upper rim 114 of tulip 102. With this arrangement, set screw 104 may be dropped into the axial bore 112 and turned, such as one quarter or one half turn, to lock it in place.

The bottom of set screw 104 may be provided with a variable curvature to accommodate different rod sizes. For example, set screw 104 may be used with both 5.5 mm and 6.2 mm diameter rods.

FIGS. 11E shows a polyaxial screw 106 configured for use with tulip 102. Screw 106 includes a threaded shank 118 and a head 120 at the proximal end of the shank. Head 120 comprises a frusto-sphere having a bearing surface 122 on its outer circumference. Screw 106 is coupled to tulip 102 by passing shank 118 through a central axial bore in the bottom of tulip 102. A spherically-curved bearing surface is provided in the interior of the bottom portion of tulip 102 for mating with bearing surface 122 of screw 106 for allowing tulip 102 to pivot in any direction relative to screw 106.

With the above configuration, screw 106 may be coupled to tulip 102 and screwed into a vertebral body using a tool mating with screw head 120. A cross bar may then be placed through the transverse bore 110 of tulip 102, and temporarily secured in place with set screw 104. Before set screw 104 is tightened against the crossbar, the orientation of the crossbar and tulip relative to screw 106 may be adjusted by allowing the bearing surface in the bottom of tulip 102 to slide relative to bearing surface 122 of screw 106. Once the desired orientation is achieved, set screw 104 is fully tightened against the cross bar, which rests on screw head 120. Accordingly, the crossbar in turn forces bearing surface 122 against the bottom of tulip 102, locking the crossbar to tulip 102 and fixing the orientation of both relative to screw 106. FIG. 11F shows a sagital screw 108 that is configured similar to polyaxial screw 106. In other embodiments, a screw shank may be integrally formed on tulip 102 with a fixed angle. The fixed angle may be zero (i.e. aligned with the longitudinal axis of the tulip), or a non-zero angle, depending on the application. A screw shank may be configured to be detachably coupled to tulip 102.

What is claimed is:

1. A pedicle screw comprising:
a shaft having a forward end, a rear end and a longitudinal axis, the forward end being configured for insertion into a bone;
a yoke attached to the rear end of the shaft, the yoke having an interior wall nearest the shaft configured to bear against a rod inserted into the yoke;
a cap configured to engage an end of the yoke opposite the shaft;
a saddle having a first side configured to mate with the cap within the yoke and an opposite second side configured to bear against the rod; and
at least one separating device coupled between the cap and the saddle, the at least one separating device configured to urge the cap and the saddle apart from one another,
wherein the yoke, the cap, the saddle and the at least one separating device cooperate to urge the rod against the interior wall of the yoke when the at least one separating device urges the cap and the saddle apart.

2. The pedicle screw of claim 1, wherein the at least one separating device comprises a set screw threadably engaged with the cap and bearing against the first side of the saddle.

3. The pedicle screw of claim 1, wherein the at least one separating device comprises a cam.

4. The pedicle screw of claim 1, wherein the cap and the saddle comprise inter-engaging features that inhibit the cap and the saddle from rotating with respect to one another.

5. The pedicle screw of claim 4, wherein the inter-engaging features comprise a dove-tail that inhibits the cap and the saddle from separating from one another.

6. The pedicle screw of claim 1, wherein the yoke is configured to slidably receive the cap and the saddle from a side of the yoke.

7. The pedicle screw of claim 1, wherein the yoke is configured to receive the cap and the saddle from the top of the yoke.

8. The pedicle screw of claim 1, wherein the cap and the yoke comprise inter-engaging detent features configured to inhibit the cap from moving laterally relative to the yoke.

9. The pedicle screw of claim 1, wherein the cap and the yoke comprise interlocking features configured to retain the cap in the yoke in a direction along the longitudinal axis of the shaft.

10. The pedicle screw of claim 9, wherein the interlocking features comprise undercut portions provided on opposite sides of the yoke for receiving mating undercut portions on the cap, wherein the undercut portions further serve to prevent opposite sides of the yoke from separating when the at least one separating device urges the cap and the saddle apart and urges the rod against the interior wall of the yoke.

11. A method of securing a rod to a pedicle screw, the method comprising:
providing a pedicle screw comprising a shaft having a forward end, a rear end and a longitudinal axis, and a yoke attached to the rear end of the shaft;
inserting the forward end of the pedicle screw into a bone;
inserting a rod into the yoke such that the rod contacts an interior wall of the yoke nearest the shaft;
inserting a block into the yoke, the block comprising a saddle, a cap and at least one separating device between the saddle and the cap, the cap comprising features for inter-engaging with the yoke, the saddle having a side opposite the cap configured to bear against the rod; and
operating the at least one separating device to cause it to move the saddle apart from the cap and against the rod, thereby clamping the rod between the saddle and the interior wall of the yoke.

12. The method of claim 11, wherein the block is inserted from a side of the yoke.

13. The method of claim 11, wherein the block is inserted from the top of the yoke.

14. The method of claim 11, wherein the at least one separating device comprises a set screw.

15. The method of claim 11, wherein the at least one separating device comprises a cam.
